(19) United States
(12) Patent Application Publication
(10) Pub. No.: US 2025/0255514 A1
(43) Pub. Date: Aug. 14, 2025

(54) BIOLOGICAL INFORMATION DETECTING APPARATUS, VEHICLE AND BED INCLUDING BIOLOGICAL INFORMATION DETECTING APPARATUS, AND BIOLOGICAL INFORMATION DETECTING METHOD

(71) Applicant: Murata Manufacturing Co., Ltd., Nagaokakyo-shi (JP)

(72) Inventor: Daichi UEKI, Nagaokakyo-shi (JP)

(73) Assignee: Murata Manufacturing Co., Ltd., Nagaokakyo-shi (JP)

(21) Appl. No.: 19/169,149

(22) Filed: Apr. 3, 2025

Related U.S. Application Data
(63) Continuation of application No. PCT/JP2023/041483, filed on Nov. 17, 2023.

(30) Foreign Application Priority Data
Dec. 2, 2022 (JP) 2022-193835

(51) Int. Cl. A61B 5/08; A61B 5/00; A61B 5/024; A61B 5/0245; G01S 13/88

(52) U.S. Cl. CPC A61B 5/0816; A61B 5/02405; A61B 5/02444; A61B 5/0245; A61B 5/6887; G01S 13/88

(57) ABSTRACT

A biological information detecting apparatus includes a reflecting member that reflects electromagnetic waves, and a radar device that detects a biological signal of a human body. The reflecting member is arranged between the human body and the radar device, and the radar device includes a transmitter that radiates an electromagnetic wave toward each of the human body and the reflecting member. The radar device also includes a first receiver that receives a first reflected wave reflected on the human body and the reflecting member, and a second receiver that receives a second reflected wave reflected on the human body.

### CROSS-REFERENCE TO RELATED APPLICATIONS

This application is a continuation of international application no. PCT/JP2023/041483, filed Nov. 17, 2023, and which claims priority to Japanese application no. 2022-193835, filed Dec. 2, 2022. The entire contents of both prior applications are hereby incorporated by reference.

### TECHNICAL FIELD

The present disclosure relates to a biological information detecting apparatus, a vehicle and a bed including the biological information detecting apparatus, and a biological information detecting method.

### BACKGROUND ART

As a biological information detecting apparatus that detects a biological signal of a human body, for example, a configuration in which two pairs of non-contact biological sensors that detect, using electromagnetic waves, biological information of a person are provided at a seat in which the person sits is disclosed. Each pair of the biological sensors is configured in such a manner that a first sensor and a second sensor that emit electromagnetic waves at different frequencies toward a person are arranged adjacent to each other. One of the first sensor and the second sensor is used to detect biological information including a noise element, and the other one of the first sensor and the second sensor is used to detect a noise element. Biological information on a human body is extracted by taking a difference corresponding to a noise element. These sensors are, for example, Doppler radars.

### SUMMARY

### Technical Problem

However, since the first sensor and the second sensor that radiate and receive electromagnetic waves at different transmission/reception frequencies are used as radio wave sensors in the above-mentioned conventional biological information detecting apparatus, the apparatus may be large in size. Furthermore, the apparatus scale of the conventional biological information detecting apparatus may be increased, and introduction cost may be increased.

The present disclosure has been designed in view of the above description, and an object of the present disclosure is to achieve a biological information detecting apparatus capable of obtaining a high-reliability biological signal of a human body while suppressing an increase of the apparatus scale and reducing the size and cost of the apparatus, a vehicle and a bed including the biological information detecting apparatus, and a biological information detecting method.

### Solution to Problem

A biological information detecting apparatus according to an aspect of the present disclosure includes a reflecting member that reflects an electromagnetic wave, and a radar device that detects a biological signal of a human body. The reflecting member is arranged between the human body and the radar device. The radar device includes a transmitter that radiates an electromagnetic wave toward each of the human body and the reflecting member, and a first receiver and a second receiver that receive a reflected wave of the electromagnetic wave. The transmitter radiates an electromagnetic wave including a first direction polarized wave component and a second direction polarized wave component that is different from the first direction polarized wave component. The reflecting member reflects the first direction polarized wave component included in the electromagnetic wave radiated from the transmitter and transmits or absorbs the second direction polarized wave component included in the electromagnetic wave radiated from the transmitter. The first receiver receives at least a first direction polarized wave component of a reflected wave reflected on the human body and the reflecting member. The second receiver receives a second direction polarized wave component of a reflected wave reflected on the human body.

With this configuration, an aspect is adopted in which the transmitter radiates an electromagnetic wave (transmission signal) including a first direction polarized wave component and a second direction polarized wave component that is different from the first direction polarized wave component, and the reflecting member reflects the first direction polarized wave component included in the electromagnetic wave radiated from the transmitter and transmits or absorbs the second direction polarized wave component included in the electromagnetic wave radiated from the transmitter. The first receiver receives at least a first direction polarized wave component of a reflected wave reflected on the human body and the reflecting member, and the second receiver receives a second direction polarized wave component of the reflected wave reflected on the human body. With this configuration, the second direction polarized wave component of the reflected wave reflected by the reflecting member can be suppressed. As a result, the accuracy of a signal calculated from the second direction polarized wave component of the reflected wave received by the second receiver can be increased.

A biological information detecting apparatus according to an aspect of the present disclosure includes a reflecting member that reflects an electromagnetic wave, a first radar device that radiates an electromagnetic wave polarized in a first direction to the reflecting member and a human body and receives a reflected wave of the electromagnetic wave, and a second radar device that radiates an electromagnetic wave polarized in a second direction that is different from the electromagnetic wave polarized in the first direction to the reflecting member and the human body and receives a reflected wave of the electromagnetic wave. The reflecting member is arranged between the human body and the first radar device, reflects the electromagnetic wave polarized in the first direction radiated from the first radar device, and transmits or absorbs the electromagnetic wave polarized in the second direction radiated from the second radar device.

With this configuration, an aspect is adopted in which the first radar device radiates an electromagnetic wave (transmission signal) polarized in a first direction to a reference surface of the reflecting member and a human body surface of a subject and receives a reflected wave of the electromagnetic wave, the second radar device radiates an electromagnetic wave polarized in a second direction that is different from the electromagnetic wave polarized in the first direction to the reflecting member and the human body and receives a reflected wave of the electromagnetic wave, and the reflecting member reflects the electromagnetic wave polarized in the first direction radiated from the first radar device and transmits or absorbs the electromagnetic wave polarized in the second direction radiated from the second radar device. With this configuration, the reflected wave of the electromagnetic wave polarized in the second direction reflected by the reflecting member can be suppressed. As a result, the accuracy of a signal calculated from the reflected wave of the electromagnetic wave polarized in the second direction transmitted and received by the second radar device can be increased.

A vehicle according to an aspect of the present disclosure includes the biological information detecting apparatus described above.

With this configuration, a vehicle capable of obtaining a high-reliability biological signal of a human body while suppressing an increase of the apparatus scale and reducing the size and cost of the apparatus can be achieved.

A bed according to an aspect of the present disclosure includes the biological information detecting apparatus described above.

With this configuration, a bed capable of obtaining a high-reliability biological signal of a human body while suppressing an increase of the apparatus scale and reducing the size and cost of the apparatus can be achieved.

A biological information detecting method according to an aspect of the present disclosure includes a first electromagnetic wave radiating step of radiating an electromagnetic wave including a first direction polarized wave component and a second direction polarized wave component that is different from the first direction polarized wave component toward a human body, a first reflected wave receiving step of receiving a reflected wave of the second direction polarized wave component, out of the first direction polarized wave component and the second direction polarized wave component included in the electromagnetic wave radiated in the first electromagnetic wave radiating step, a second electromagnetic wave radiating step of radiating an electromagnetic wave including a first direction polarized wave component and a second direction polarized wave component toward a reflecting member that reflects an electromagnetic wave, a second reflected wave receiving step of receiving a reflected wave of the first direction polarized wave component, out of the first direction polarized wave component and the second direction polarized wave component included in the electromagnetic wave radiated in the second electromagnetic wave radiating step, a displacement signal generating step of generating, from the reflected wave received in the first reflected wave receiving step, a second displacement signal and generating, from the reflected wave received in the second reflected wave receiving step, a first displacement signal, and a biological signal generating step of isolating the first displacement signal from the second displacement signal, which is generated in the displacement signal generating step, to generate a biological signal of the human body.

With this configuration, in the first electromagnetic wave radiating step, an electromagnetic wave (transmission signal) including a first direction polarized wave component and a second direction polarized wave component that is different from the first direction polarized wave component is radiated toward the human body. In the first reflected wave receiving step, a reflected wave of the second direction polarized wave component, out of the first direction polarized wave component and the second direction polarized wave component included in the electromagnetic wave radiated in the first electromagnetic wave radiating step, is received. Furthermore, in the second electromagnetic wave radiating step, an electromagnetic wave including a first direction polarized wave component and a second direction polarized wave component is radiated toward the reflecting member. In the second reflected wave receiving step, a reflected wave of the first direction polarized wave component, out of the first direction polarized wave component and the second direction polarized wave component included in the electromagnetic wave radiated in the second electromagnetic wave radiating step, is received. In the displacement signal generating step, a second displacement signal is generated from the reflected wave received in the first reflected wave receiving step, and a first displacement signal is generated from the reflected wave received in the second reflected wave receiving step. In the biological signal generating step, the first displacement signal is isolated from the second displacement signal, which is generated in the displacement signal generating step, and a biological signal of the human body is generated. With this configuration, the second direction polarized wave component of the reflected wave reflected by the reflecting member is suppressed, and the accuracy of the second displacement signal generated based on the second direction polarized wave component of the reflected wave can be increased. In other words, a second displacement signal in which the impact of a reflected wave component from the reflecting member is suppressed can be obtained. Thus, a high-reliability biological signal can be obtained.

A biological information detecting method according to an aspect of the present disclosure includes an electromagnetic wave radiating step of radiating an electromagnetic wave polarized in a first direction toward a reflecting member that reflects an electromagnetic wave and radiating an electromagnetic wave polarized in a second direction that is different from the electromagnetic wave polarized in the first direction toward a human body, a reflected wave receiving step of receiving a reflected wave of the electromagnetic wave polarized in the first direction radiated in the electromagnetic wave radiating step and receiving a reflected wave of the electromagnetic wave polarized in the second direction radiated in the electromagnetic wave radiating step, a displacement signal generating step of generating, from the reflected wave of the electromagnetic wave polarized in the second direction received in the reflected wave receiving step, a second displacement signal and generating, from the reflected wave of the electromagnetic wave polarized in the first direction received in the reflected wave receiving step, a first displacement signal, and a biological signal generating step of isolating the first displacement signal from the second displacement signal, which is generated in the displacement signal generating step, to generate a biological signal of the human body.

With this configuration, in the electromagnetic wave radiating step, an electromagnetic wave polarized in a first direction is radiated toward the reflecting member, and an electromagnetic wave polarized in a second direction that is different from the electromagnetic wave polarized in the first direction is radiated toward the human body. In the reflected wave receiving step, a reflected wave of the electromagnetic wave polarized in the first direction radiated in the electromagnetic wave radiating step is received, and a reflected wave of the electromagnetic wave polarized in the second direction radiated in the electromagnetic wave radiating step is received. In the displacement signal generating step, a second displacement signal is generated from the reflected wave of the electromagnetic wave polarized in the second direction received in the reflected wave receiving step, and a first displacement signal is generated from the reflected wave of the electromagnetic wave polarized in the first direction received in the reflected wave receiving step. In the biological signal generating step, the first displacement signal is isolated from the second displacement signal, which is generated in the displacement signal generating step, and a biological signal of the human body is generated. With this configuration, the second direction polarized wave component of the reflected wave reflected by the reflecting member is suppressed, and the accuracy of the second displacement signal generated based on the second direction polarized wave component of the reflected wave can be increased. In other words, a second displacement signal in which the impact of a reflected wave component from the reflecting member is suppressed can be obtained. Thus, a high-reliability biological signal can be obtained.

### Advantageous Effects

According to the present disclosure, a biological information detecting apparatus capable of obtaining a high-reliability biological signal of a human body while suppressing an increase of the apparatus scale and reducing the size and cost of the apparatus, a vehicle and a bed including the biological information detecting apparatus, and a biological information detecting method can be achieved.

### BRIEF DESCRIPTION OF DRAWINGS

FIG. 1 is a block diagram illustrating a schematic configuration of a biological information detecting apparatus according to Exemplary Embodiment 1.

FIG. 2 is a side view illustrating an application example of the case where the biological information detecting apparatus according to Exemplary Embodiment 1 is applied as a driver monitoring system.

FIG. 3 is a schematic diagram illustrating an example of the shape of a reflecting member.

FIG. 4A is a schematic diagram illustrating a first example of an antenna surface of a dielectric substrate that configures a radar device.

FIG. 4B is a schematic diagram illustrating a second example of the antenna surface of the dielectric substrate that configures the radar device.

FIG. 5 is a diagram illustrating combination examples of a polarized wave of an electromagnetic wave reflected on a reference surface of a reflecting member, a polarized wave of an electromagnetic wave transmitted from a transmitter, a polarized wave of an electromagnetic wave received by a first receiver, and a polarized wave of an electromagnetic wave received by a second receiver in the biological information detecting apparatus according to Exemplary Embodiment 1.

FIG. 6A is a diagram illustrating an example of a second displacement signal.

FIG. 6B is a diagram illustrating an example of a first displacement signal.

FIG. 6C is a diagram illustrating an example of a biological signal.

FIG. 7 is a flowchart illustrating an example of a biological information detecting process by the biological information detecting apparatus.

FIG. 8 is a block diagram illustrating a schematic configuration of a biological information detecting apparatus according to Exemplary Embodiment 2.

FIG. 9 is a diagram illustrating combination examples of a polarized wave of an electromagnetic wave reflected on a reference surface of a reflecting member, a polarized wave of an electromagnetic wave transmitted and received by a first radar device, and a polarized wave of an electromagnetic wave transmitted and received by a second radar device in the biological information detecting apparatus according to Exemplary Embodiment 2.

FIG. 10 is a perspective view illustrating an arrangement example of the case where a biological information detecting apparatus according to the present disclosure is applied to a vehicle.

FIG. 11 is a perspective view illustrating an application example of the case where a biological information detecting apparatus according to the present disclosure is applied to a bed in a medical facility.

### DESCRIPTION OF EXEMPLARY EMBODIMENTS

Hereinafter, a biological information detecting apparatus according to an exemplary embodiment, a vehicle and a bed including the biological information detecting apparatus, and a biological information detecting method will be described in detail with reference to drawings. The present disclosure is not intended to be limited by the exemplary embodiments described below.

### Exemplary Embodiment 1

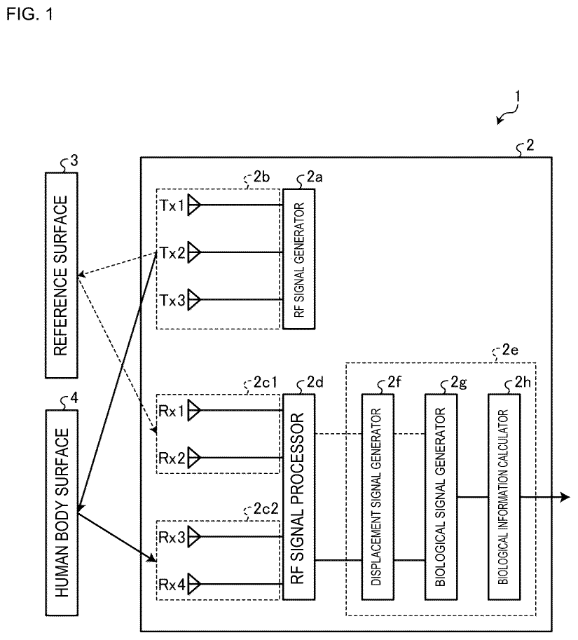

FIG. 1 is a block diagram illustrating a schematic configuration of a biological information detecting apparatus according to Exemplary Embodiment 1. A biological information detecting apparatus 1 according to Exemplary Embodiment 1 includes a radar device 2 and a reflecting member 3.

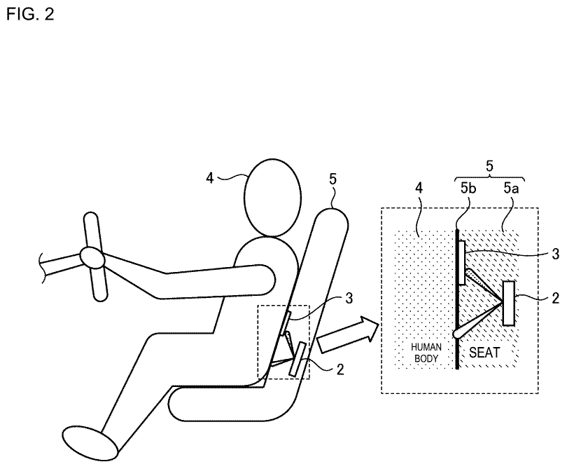

FIG. 2 is a side view illustrating an application example of the case where the biological information detecting apparatus according to Exemplary Embodiment 1 is applied as a driver monitoring system. The biological information detecting apparatus 1 is applied to, for example, a driver monitoring system (DMS) illustrated in FIG. 2, and is installed inside a seat 5 of a vehicle in which a driver as a subject 4 sits.

As illustrated in an enlarged part inside a frame indicated by a broken line in FIG. 2, the radar device 2 is installed inside an inner material 5a of the seat 5. With the biological information detecting apparatus 1 installed as mentioned above, a variation in the distance from the radar device 2 to a body surface of the subject 4 to which an electromagnetic wave is radiated is detected as a body surface displacement by the biological information detecting apparatus 1. Based on the body surface displacement, vital signs, such as the heart rate, heart rate variability, respiration rate, and depth of respiration, of the subject 4 who is driving the vehicle are detected by the biological information detecting apparatus 1.

The reflecting member 3 is installed between the human body of the subject 4 and the radar device 2. Specifically, for example, an aspect is illustrated in which the reflecting member 3 is installed at the back of a surface material 5b of the seat 5 that is in contact with the human body of the subject 4, that is, near the inner material 5a. The reflecting member 3 is made of a material that reflects an electromagnetic wave radiated (emitted) from the radar device 2. For example, the reflecting member 3 is arranged in direct contact or indirect contact with the human body of the subject 4. The reflecting member 3 may be arranged at a member that picks up body motion of the subject 4. For example, as in this exemplary embodiment, the reflecting member 3 is arranged at a back section, a sitting section, or the like of the seat 5.

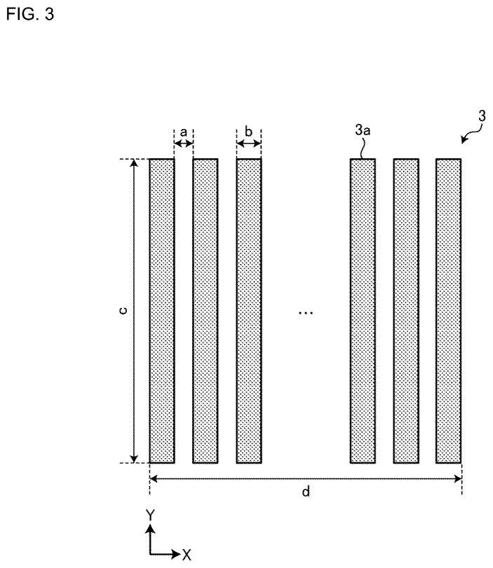

FIG. 3 is a schematic diagram illustrating an example of the shape of a reflecting member. In the example illustrated in FIG. 3, in the reflecting member 3, a plurality of reflectors 3a extending in a Y direction are aligned in an X direction. Each of the reflectors 3a is made of a material reflecting a radio wave, such as metal. In the present disclosure, the wavelength of an electromagnetic wave transmitted and received by the radar device 2 is represented by λ. The gap a between the reflectors 3a is set to, for example, 1λ. The width b in the X direction of each of the reflectors 3a is set to, for example, 0.25λ. The length c in the Y direction of each of the reflectors 3a is set to, for example, 10λ. The width d in the X direction of the reflecting member 3 including the plurality of reflectors 3a is set to, for example, 10λ. Sizes of the gap a between the reflectors 3a, the width b in the X direction of each of the reflectors 3a, the length c in the Y direction of each of the reflectors 3a, and the width d in the X direction of the reflecting member 3 are examples and are optimized by the positional relationship, distance, and the like between the reflecting member 3 and the radar device 2.

In an aspect illustrated in FIG. 3, the reflecting member 3 transmits radio waves with the electric field in the X direction and reflects radio waves with the electric field in the Y direction. Hereinafter, radio waves with the electric field in the X direction will also be referred to as “horizontally polarized waves” and radio waves with the electric field in the Y direction will also be referred to as “vertically polarized waves”. In other words, in the aspect illustrated in FIG. 3, the reflecting member 3 transmits a horizontally polarized wave component and reflects a vertically polarized wave component.

Taking into consideration the comfort when the subject 4 sits in the seat 5, it is desirable that the material of each of the reflectors 3a be electrically conductive, such as metallic foil or conductive fiber, and follow a change in the shape of the surface in contact with a human body. However, each of the reflectors 3a is not necessarily made of the material mentioned above. Each of the reflectors 3a may be made of a hard metal plate as long as it reflects electromagnetic waves. Furthermore, the material may be conductive fiber reinforced plastic, a member plated with a conductive material, a member coated with a conductive coating, or a member attached with a conductive tape.

The radar device 2 is configured as a module including an RF signal generator 2a, a transmitter 2b, a first receiver 2c1, a second receiver 2c2, an RF (high frequency) signal processor 2d, and a calculator 2e. These units are implemented by a software control process of a microcomputer, a hardware configuration of an electronic circuit, or both the software control process of the microcomputer and the hardware configuration of the electronic circuit.

The RF signal generator 2a, the RF signal processor 2d, and the calculator 2e are configured as an integrated circuit (IC) and are, for example, arranged at the back of an antenna surface of a dielectric substrate. Electromagnetic waves are modulated by the radar device 2 in a Doppler method, a frequency modulated continuous wave radar (FMCW) method, a pulse modulation method, or the like. A method in which the radar device 2 modulates electromagnetic waves is not limited to the modulation methods mentioned above as long as the distance to an object can be measured.

The RF signal generator 2a generates a chirp signal as a transmission signal. The transmitter 2b includes a plurality of transmission antennas Tx that radiate electromagnetic waves to the human body of the subject 4 and the reflecting member 3. In FIG. 1, an example in which transmission antennas Tx1, Tx2, and Tx3 are arrayed is illustrated. In the transmitter 2b, the number of arrays (the number of transmission antennas Tx configuring the transmitter 2b) can be increased or decreased according to the gain required.

The transmitter 2b performs beamforming on a transmission signal generated by the RF signal generator 2a and radiates the transmission signal, as electromagnetic waves, from the plurality of transmission antennas Tx toward the human body of the subject 4 and the reflecting member 3. In this exemplary embodiment, electromagnetic waves radiated from the transmitter 2b are described as radio waves. However, electromagnetic waves widely include sound waves, light waves, and the like.

The first receiver 2c1 includes a plurality of reception antennas Rx that receive reflected waves reflected on the human body surface of the subject 4 and reflected waves reflected on the reference surface of the reflecting member 3. In FIG. 1, an example in which reception antennas Rx1 and Rx2 are arrayed is illustrated. In the first receiver 2c1, the number of arrays (the number of reception antennas Rx configuring the first receiver 2c1) can be increased or decreased according to the gain required.

The second receiver 2c2 includes a plurality of reception antennas Rx that receive reflected waves reflected on the human body surface of the subject 4. In FIG. 1, an example in which reception antennas Rx3 and Rx4 are arrayed is illustrated. In the second receiver 2c2, the number of arrays (the number of reception antennas Rx configuring the second receiver 2c2) can be increased or decreased according to the gain required.

The transmitter 2b, the first receiver 2c1, and the second receiver 2c2 are pattern antennas provided on a surface of the dielectric substrate configuring the radar device 2, more specifically, the surface of the dielectric substrate near the reference surface of the reflecting member 3 and the human body surface of the subject 4. Hereinafter, the surface of the dielectric substrate on which the transmitter 2b, the first receiver 2c1, and the second receiver 2c2 are provided will also be referred to as an “antenna surface”.

As the material of the dielectric substrate configuring the radar device 2, for example, a low temperature co-fired ceramics (LTCC) multilayer substrate, a multilayer resin substrate formed by laminating a plurality of resin layers made of a resin such as epoxy or polyimide, a multilayer resin substrate formed by laminating a plurality of resin layers made of liquid crystal polymer (LCP) with a lower permittivity, a multilayer resin substrate formed by laminating a plurality of resin layers made of a fluorine-based resin, a ceramics multilayer substrate (excluding a low temperature co-fired ceramics multilayer substrate), or the like is illustrated.

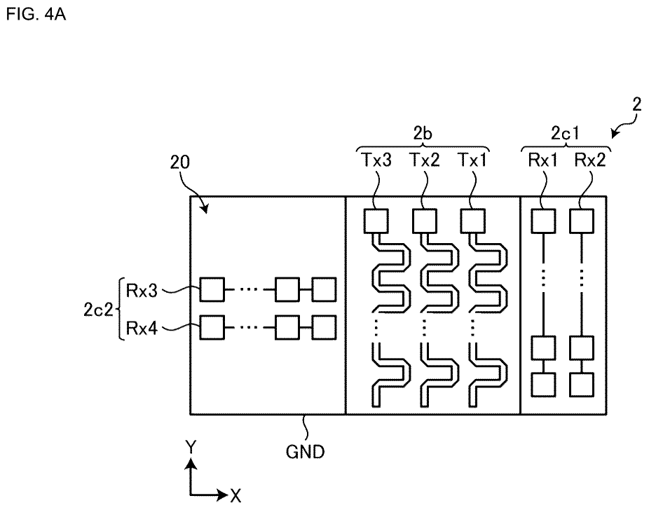

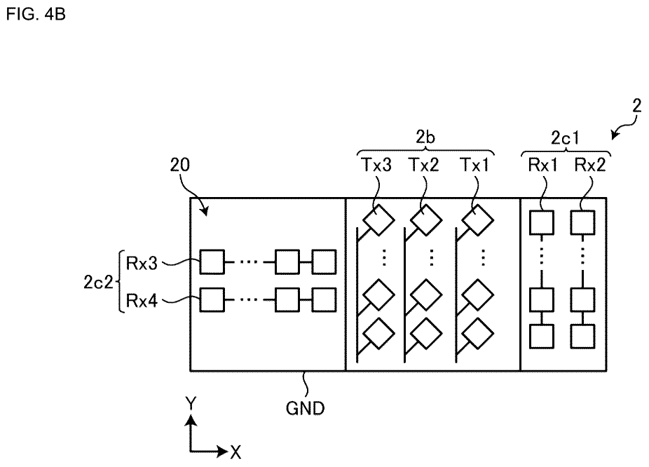

FIG. 4A is a schematic diagram illustrating a first example of the antenna surface of the dielectric substrate configuring the radar device. FIG. 4B is a schematic diagram illustrating a second example of the antenna surface of the dielectric substrate configuring the radar device. As illustrated in FIGS. 4A and 4B, on the antenna surface of a dielectric substrate 20, the transmitter 2b, the first receiver 2c1, and the second receiver 2c2 are each surrounded by a GND pattern.

In an aspect illustrated in FIG. 4A, the transmitter 2b radiates, as a circularly polarized wave, an electromagnetic wave to the reference surface of the reflecting member 3 or the human body surface of the subject 4. The reflecting member 3 in the aspect illustrated in FIG. 3 reflects a vertically polarized wave component of the circularly polarized wave radiated from the transmitter 2b and transmits a component of the circularly polarized wave that includes a horizontally polarized wave component but does not include the vertically polarized wave component. The first receiver 2c1 receives a vertically polarized wave component of a reflected wave reflected on the reference surface of the reflecting member 3 or the human body surface of the subject 4. The second receiver 2c2 receives a horizontally polarized wave component of the reflected wave reflected on the human body surface of the subject 4.

In an aspect illustrated in FIG. 4B, the transmitter 2b radiates, as a diagonally polarized wave, which is slanted in the X direction and the Y direction (for example, a tilt θ=45 degrees with respect to the X direction and the Y direction), an electromagnetic wave to the reference surface of the reflecting member 3 or the human body surface of the subject 4. The reflecting member 3 in the aspect illustrated in FIG. 3 reflects a vertically polarized wave component of the diagonally polarized wave radiated from the transmitter 2b and transmits a component of the diagonally polarized wave that includes a horizontally polarized wave component but does not include the vertically polarized wave component. The first receiver 2c1 receives a vertically polarized wave component of a reflected wave reflected on the reference surface of the reflecting member 3 or the human body surface of the subject 4. The second receiver 2c2 receives a horizontally polarized wave component of the reflected wave reflected on the human body surface of the subject 4.

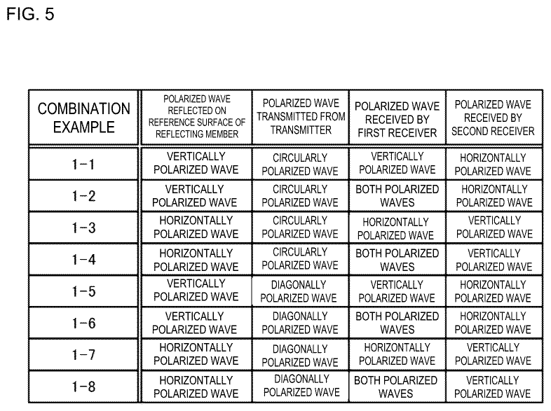

Although an aspect in which the reflecting member 3 reflects a vertically polarized wave component of an electromagnetic wave (transmission signal) radiated from the transmitter 2b, the first receiver 2c1 receives a vertically polarized wave component of a reflected wave reflected on the reference surface of the reflecting member 3 or the human body surface of the subject 4, and the second receiver 2c2 receives a horizontally polarized wave component of the reflected wave reflected on the human body surface of the subject 4 is illustrated in FIGS. 3, 4A, and 4B, the present disclosure is not limited to this aspect. FIG. 5 is a diagram illustrating combination examples of a polarized wave of an electromagnetic wave reflected on the reference surface of the reflecting member, a polarized wave of an electromagnetic wave transmitted from the transmitter, a polarized wave of an electromagnetic wave received by the first receiver, and a polarized wave of an electromagnetic wave received by the second receiver in the biological information detecting apparatus according to Exemplary Embodiment 1.

More specifically, a combination example 1-1 is a combination example of the reflecting member 3 illustrated in FIG. 3 and the transmitter 2b, the first receiver 2c1, and the second receiver 2c2 illustrated in FIG. 4A. That is, in an aspect of the combination example 1-1, the transmitter 2b radiates, as a circularly polarized wave, an electromagnetic wave to the reference surface of the reflecting member 3 or the human body surface of the subject 4. The reflecting member 3 reflects a vertically polarized wave component of the circularly polarized electromagnetic wave radiated from the transmitter 2b, and transmits a component of the circularly polarized electromagnetic wave that includes a horizontally polarized wave component but does not include the vertically polarized wave component. The first receiver 2c1 receives a vertically polarized wave component of a reflected wave reflected on the reference surface of the reflecting member 3 or the human body surface of the subject 4. The second receiver 2c2 receives a horizontally polarized wave component of the reflected wave reflected on the human body surface of the subject 4.

In an aspect of a combination example 1-2, the transmitter 2b radiates, as a circularly polarized wave, an electromagnetic wave to the reference surface of the reflecting member 3 or the human body surface of the subject 4. The reflecting member 3 reflects a vertically polarized wave component of the circularly polarized electromagnetic wave radiated from the transmitter 2b, and transmits a component of the circularly polarized electromagnetic wave that includes a horizontally polarized wave component but does not include the vertically polarized wave component. The first receiver 2c1 receives both polarized wave components (for example, a circularly polarized wave) including both of a vertically polarized wave component and a horizontally polarized wave component of a reflected wave reflected on the reference surface of the reflecting member 3 or the human body surface of the subject 4. The second receiver 2c2 receives the horizontally polarized wave component of the reflected wave reflected on the human body surface of the subject 4.

In an aspect of a combination example 1-3, the transmitter 2b radiates, as a circularly polarized wave, an electromagnetic wave to the reference surface of the reflecting member 3 or the human body surface of the subject 4. The reflecting member 3 reflects a horizontally polarized wave component of the circularly polarized electromagnetic wave radiated from the transmitter 2b, and transmits a component of the circularly polarized electromagnetic wave that includes a vertically polarized wave component but does not include the horizontally polarized wave component. The first receiver 2c1 receives a horizontally polarized wave component of a reflected wave reflected on the reference surface of the reflecting member 3 or the human body surface of the subject 4. The second receiver 2c2 receives a vertically polarized wave component of the reflected wave reflected on the human body surface of the subject 4.

In an aspect of a combination example 1-4, the transmitter 2b radiates, as a circularly polarized wave, an electromagnetic wave to the reference surface of the reflecting member 3 or the human body surface of the subject 4. The reflecting member 3 reflects a horizontally polarized wave component of the circularly polarized electromagnetic wave radiated from the transmitter 2b, and transmits a component of the circularly polarized electromagnetic wave that includes a vertically polarized wave component but does not include the horizontally polarized wave component. The first receiver 2c1 receives both polarized wave components (for example, a circularly polarized wave) including both of a horizontally polarized wave component and a vertically polarized wave component of a reflected wave reflected on the reference surface of the reflecting member 3 or the human body surface of the subject 4. The second receiver 2c2 receives the vertically polarized wave component of the reflected wave reflected on the human body surface of the subject 4.

A combination example 1-5 is a combination example of the reflecting member 3 illustrated in FIG. 3 and the transmitter 2b, the first receiver 2c1, and the second receiver 2c2 illustrated in FIG. 4B. That is, in an aspect of the combination example 1-5, the transmitter 2b radiates, as a diagonally polarized wave, an electromagnetic wave to the reference surface of the reflecting member 3 or the human body surface of the subject 4. The reflecting member 3 reflects a vertically polarized wave component of the diagonally polarized electromagnetic wave radiated from the transmitter 2b, and transmits a component of the diagonally polarized electromagnetic wave that includes a horizontally polarized wave component but does not include the vertically polarized wave component. The first receiver 2c1 receives a vertically polarized wave component of a reflected wave reflected on the reference surface of the reflecting member 3 or the human body surface of the subject 4. The second receiver 2c2 receives a horizontally polarized wave component of the reflected wave reflected on the human body surface of the subject 4.

In an aspect of a combination example 1-6, the transmitter 2b radiates, as a diagonally polarized wave, an electromagnetic wave to the reference surface of the reflecting member 3 or the human body surface of the subject 4. The reflecting member 3 reflects a vertically polarized wave component of the diagonally polarized electromagnetic wave radiated from the transmitter 2b, and transmits a component of the diagonally polarized electromagnetic wave that includes a horizontally polarized wave component but does not include the vertically polarized wave component. The first receiver 2c1 receives both polarized wave components (for example, a circularly polarized wave) including both of a vertically polarized wave component and a horizontally polarized wave component of a reflected wave reflected on the reference surface of the reflecting member 3 or the human body surface of the subject 4. The second receiver 2c2 receives the horizontally polarized wave component of the reflected wave reflected on the human body surface of the subject 4.

In an aspect of a combination example 1-7, the transmitter 2b radiates, as a diagonally polarized wave, an electromagnetic wave to the reference surface of the reflecting member 3 or the human body surface of the subject 4. The reflecting member 3 reflects a horizontally polarized wave component of the diagonally polarized electromagnetic wave radiated from the transmitter 2b, and transmits a component of the diagonally polarized electromagnetic wave that includes a vertically polarized wave component but does not include the horizontally polarized wave component. The first receiver 2c1 receives a horizontally polarized wave component of a reflected wave reflected on the reference surface of the reflecting member 3 or the human body surface of the subject 4. The second receiver 2c2 receives a vertically polarized wave component of the reflected wave reflected on the human body surface of the subject 4.

In an aspect of a combination example 1-8, the transmitter 2b radiates, as a diagonally polarized wave, an electromagnetic wave to the reference surface of the reflecting member 3 or the human body surface of the subject 4. The reflecting member 3 reflects a horizontally polarized wave component of the diagonally polarized electromagnetic wave radiated from the transmitter 2b, and transmits a component of the diagonally polarized electromagnetic wave that includes a vertically polarized wave component but does not include the horizontally polarized wave component. The first receiver 2c1 receives both polarized wave components (for example, a circularly polarized wave) including both of a horizontally polarized wave component and a vertically polarized wave component of a reflected wave reflected on the reference surface of the reflecting member 3 or the human body surface of the subject 4. The second receiver 2c2 receives the vertically polarized wave component of the reflected wave reflected on the human body surface of the subject 4.

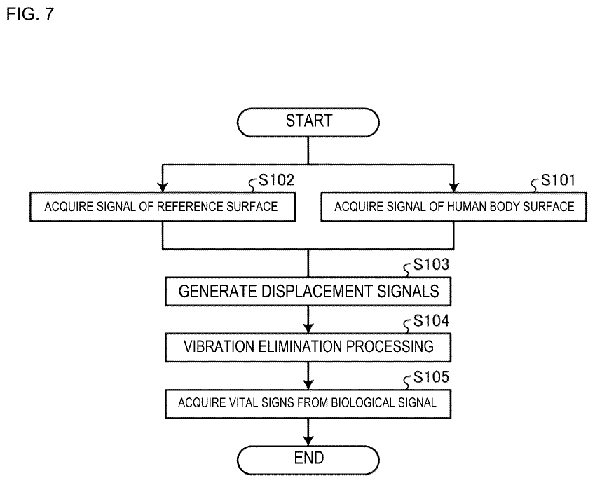

Out of the combination examples described above, in the aspects of the combination examples 1-2, 1-4, 1-6, and 1-8, the first receiver 2c1 receives both the horizontally polarized wave component and the vertically polarized wave component of the reflected wave. However, when a first IF signal, which is a signal of the reference surface of the reflecting member 3, is acquired in first IF signal acquiring step S102 of a biological information detecting process (see FIG. 7), which will be described later, since an electromagnetic wave is radiated from the transmission antenna Tx of the transmitter 2b toward the reference surface of the reflecting member 3, a polarized wave reflected from the reflecting member 3 has priority, as a polarized wave received by the first receiver 2c1, over reflection from the human body surface of the subject 4. Therefore, even in the aspects of the combination examples 1-2, 1-4, 1-6, and 1-8 in which the first receiver 2c1 receives both the horizontally polarized wave component and the vertically polarized wave component of the reflected wave, the impact of reflection from the human body surface of the subject 4 is extremely small as the first IF signal acquired in the first IF signal acquiring step S102.

Furthermore, the reflecting member 3 including the reference surface is not limited to the aspect illustrated in FIG. 3. Specifically, an aspect may be adopted in which the reflecting member 3 including the reference surface is, for example, a slot antenna, a dipole array, a patch antenna array, or the like, and an aspect may be adopted in which the reflecting member 3 is terminated at a predetermined impedance so that components other than a polarized wave component received by the first receiver 2c1 can be absorbed.

As described above, an aspect may be adopted in which the transmitter 2b radiates an electromagnetic wave (transmission signal) including a first direction polarized wave and a second direction polarized wave that is different from the first direction polarized wave, the reflecting member 3 reflects the first direction polarized wave included in the electromagnetic wave radiated from the transmitter 2b and transmits or absorbs the second direction polarized wave included in the electromagnetic wave radiated from the transmitter 2b, the first receiver 2c1 receives at least a first direction polarized wave of a reflected wave reflected on the human body surface of the subject 4 or the reference surface of the reflecting member 3, and the second receiver 2c2 receives a second direction polarized wave reflected on the human body surface of the subject 4. In the description provided below, an example in which the combination example 1-1 illustrated in FIG. 5, that is, aspects of the shape of the reflecting member 3 illustrated in FIG. 3 and the first example of the antenna surface of the dielectric substrate 20 configuring the radar device 2 illustrated in FIG. 4A, are adopted will be described.

The RF signal processor 2d receives the reflected wave received by the first receiver 2c1 to calculate the first IF signal, converts, with an AD converter, the calculated first IF signal into a digital signal, and outputs the digital signal to the calculator 2e.

Furthermore, the RF signal processor 2d receives the reflected wave received by the second receiver 2c2 to calculate a second IF signal, converts, with the AD converter, the calculated second IF signal into a digital signal, and outputs the digital signal to the calculator 2e.

The calculator 2e includes a displacement signal generator 2f, a biological signal generator 2g, and a biological information calculator 2h.

The displacement signal generator 2f performs fast Fourier transform (FFT) on a signal received from the RF signal processor 2d. More specifically, the displacement signal generator 2f generates, based on the first IF signal calculated from the reflected wave received by the first receiver 2c1, a first displacement signal. Furthermore, the displacement signal generator 2f generates, based on the second IF signal calculated from the reflected wave received by the second receiver 2c2, a second displacement signal.

The biological signal generator 2g isolates the first displacement signal, which is generated based on the first IF signal, from the second displacement signal, which is generated based on the second IF signal, to generate a biological signal of the human body of the subject 4.

As a technique for isolating the first displacement signal from the second displacement signal to generate the biological signal of the human body of the subject 4, for example, a technique using an adaptive filter that adopts an algorithm such as a least mean square (LMS) is illustrated. The technique for isolating the first displacement signal from the second displacement signal is not limited to the technique mentioned above. For example, there may be an aspect in which blind source separation (BSS) such as independent component analysis (ICA), independent vector analysis (IVA), or independent low-rank matrix analysis (ILRMA) is used. The present disclosure is not intended to be limited by a technique for isolating the first displacement signal from the second displacement signal.

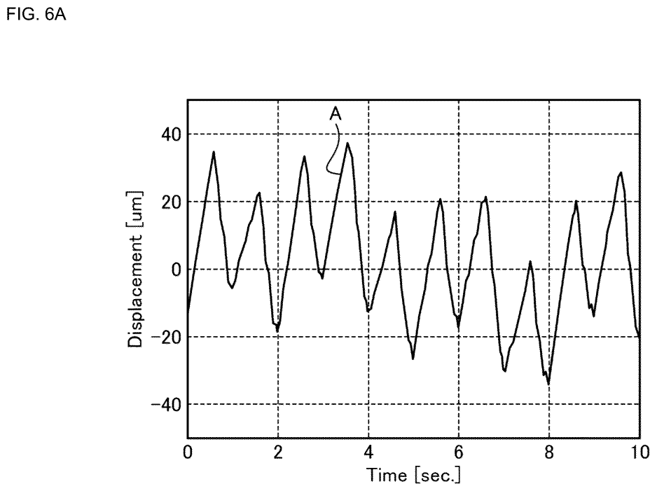

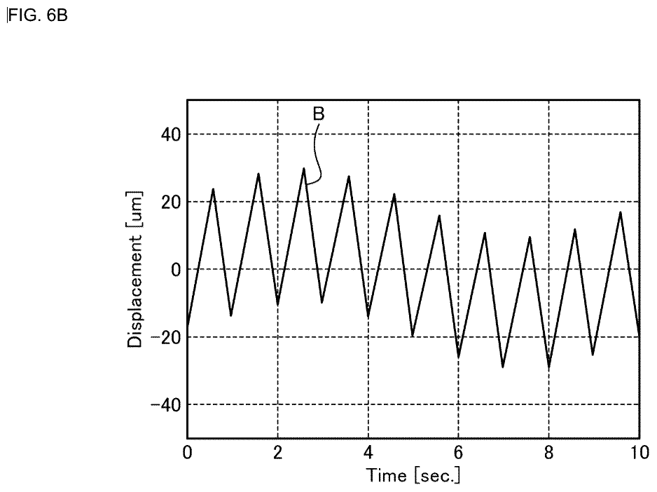

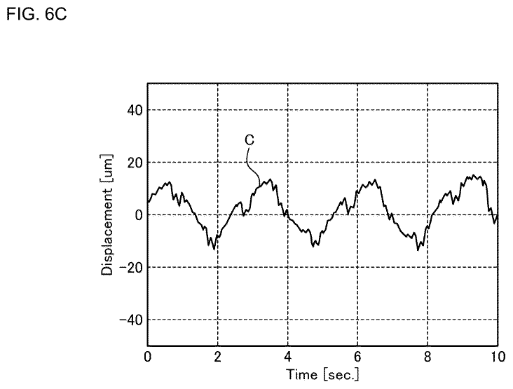

FIG. 6A is a diagram illustrating an example of the second displacement signal. FIG. 6B is a diagram illustrating an example of the first displacement signal. FIG. 6C is a diagram illustrating an example of the biological signal. In FIGS. 6A, 6B, and 6C, the horizontal axis represents time [sec], and the vertical axis represents the amount of displacement of each signal [μm]. The amount of displacement of each signal is expressed by defining the average in a time section as “0 [μm]”.

The first displacement signal is generated based on the first IF signal calculated from the reflected wave reflected on the reference surface of the reflecting member 3 and received by the first receiver 2c1. The first displacement signal is, for example, a component such as vibration caused by road noise or a behavior of the vehicle, and is a noise component (see a waveform B in FIG. 6B) with respect to a biological signal to be detected by the biological information detecting apparatus 1 according to the present disclosure.

In contrast, the second displacement signal is generated based on the second IF signal calculated from the reflected wave reflected on the human body surface of the subject 4 and received by the second receiver 2c2. The second displacement signal is a signal component (see a waveform A in FIG. 6A) in which a first displacement signal B illustrated in FIG. 6B is superimposed on a biological signal to be detected by the biological information detecting apparatus 1 according to the present disclosure.

Thus, by isolating the first displacement signal B, which is a noise component, from a second displacement signal A on which the first displacement signal B is superimposed, a biological signal to be detected by the biological information detecting apparatus 1 according to the present disclosure can be extracted (see a waveform C in FIG. 6C).

The biological information calculator 2h calculates, as biological information, vital signs, such as the heart rate, heart rate variability, respiration rate, and depth of respiration, of the human body of the subject 4 from the biological signal generated by the biological signal generator 2g.

A biological information detecting process by the biological information detecting apparatus 1 according to Exemplary Embodiment 1 will be described with reference to FIG. 7. FIG. 7 is a flowchart illustrating an example of the biological information detecting process by the biological information detecting apparatus.

First, the biological information detecting apparatus 1 according to Exemplary Embodiment 1 performs, at the same time, second IF signal acquiring step S101 for acquiring the second IF signal, which is a signal of the human body surface of the subject 4, and the first IF signal acquiring step S102 for acquiring the first IF signal, which is a signal of the reference surface of the reflecting member 3. Specifically, the second IF signal acquiring step S101 and the first IF signal acquiring step S102 are performed, for example, in such a manner that switching between the second IF signal acquiring step S101 and the first IF signal acquiring step S102 is performed every time sampling is performed by the calculator 2e. Furthermore, the RF signal generator 2a switches the radiation direction of an electromagnetic wave from the transmission antenna Tx of the transmitter 2b in synchronization with the sampling timing of the calculator 2e. More specifically, at the first sampling timing, an electromagnetic wave is radiated to the human body surface of the subject 4. At the second sampling timing following the first sampling timing, an electromagnetic wave is radiated to the reference surface of the reflecting member 3. In other words, the second IF signal acquiring step S101 and the first IF signal acquiring step S102 are performed in a time division manner.

The second IF signal acquiring step S101 includes a first electromagnetic wave radiating step and a first reflected wave receiving step. In the first electromagnetic wave radiating step, the transmitter 2b radiates a circularly polarized electromagnetic wave toward the human body of the subject 4. In the first reflected wave receiving step, the second receiver 2c2 receives, out of circularly polarized wave components of the electromagnetic wave radiated in the first electromagnetic wave radiating step, a horizontally polarized wave component of a reflected wave reflected on the human body surface of the subject 4, as a human body surface signal.

Furthermore, the first IF signal acquiring step S102 includes a second electromagnetic wave radiating step and a second reflected wave receiving step. In the second electromagnetic wave radiating step, the transmitter 2b radiates a circularly polarized electromagnetic wave toward the reflecting member 3. In the second reflected wave receiving step, the first receiver 2c1 receives, out of circularly polarized wave components of the electromagnetic wave radiated in the second electromagnetic wave radiating step, a vertically polarized wave component of a reflected wave reflected on the reference surface of the reflecting member 3, as a reference surface signal.

Next, the biological information detecting apparatus 1 performs displacement signal generating step S103. In the displacement signal generating step S103, the displacement signal generator 2f generates, from the second IF signal acquired in the second IF signal acquiring step S101, a second displacement signal indicating displacement of the body surface of the human body of the subject 4. Furthermore, the displacement signal generator 2f generates, from the first IF signal acquired in the first IF signal acquiring step S102, a first displacement signal indicating displacement of the reference surface of the reflecting member 3.

Next, the biological information detecting apparatus 1 performs biological signal generating step S104 (vibration elimination processing). In the biological signal generating step S104, the biological signal generator 2g isolates the first displacement signal, which is generated in the displacement signal generating step S103, from the second displacement signal, which is generated in a similar manner in the displacement signal generating step S103. By the biological signal generating step S104, for example, a component such as vibration caused by road noise or a behavior of the vehicle is suppressed, and a biological signal of the human body of the subject 4, which is to be detected by the biological information detecting apparatus 1 according to the present disclosure, is generated. Specifically, for example, a biological signal C illustrated in FIG. 6C includes vital signs such as respiration and heart rate of the subject 4.

Next, the biological information detecting apparatus 1 performs vital sign acquiring step S105. In the vital sign acquiring step S105, the biological information calculator 2h acquires vital signs of the human body of the subject 4 from the biological signal generated by the biological signal generator 2g in the biological signal generating step S104. Detailed description of a technique for acquiring vital signs is omitted here. The present disclosure is not intended to be limited by a technique for acquiring vital signs.

With the biological information detecting apparatus 1 and the biological information detecting method according to Exemplary Embodiment 1 described above, by the RF signal generator 2a, the transmitter 2b, the first receiver 2c1, the second receiver 2c2, the RF (high frequency) signal processor 2d, and the displacement signal generator 2f that are included inside the one radar device 2, electromagnetic waves are radiated and received (output and input) to and from the human body of the subject 4 and the reflecting member 3, and a second displacement signal indicating displacement of the body surface of the human body of the subject 4 and a first displacement signal indicating displacement of the reference surface of the reflecting member 3 are acquired. Then, by the biological signal generator 2g, the first displacement signal is isolated from the acquired second displacement signal, and a biological signal of the human body, in which a noise component superimposed on the second displacement signal is suppressed, is generated.

Thus, unlike a conventional biological information detecting apparatus in which a first sensor and a second sensor that radiate and receive electromagnetic waves at different frequencies are used as radio wave sensors, a biological signal of a human body in which a noise component is suppressed can be obtained by using the one radar device 2 as a radio wave sensor. Hence, with the biological information detecting apparatus 1 according to Exemplary Embodiment 1, an increase of the apparatus scale of the biological information detecting apparatus 1 can be suppressed, and reductions in the size and cost of the biological information detecting apparatus 1 can thus be achieved.

If an aspect is adopted in which the reflecting member 3 reflects all the polarized wave components of electromagnetic waves radiated from the transmitter 2b and the first receiver 2c1 and the second receiver 2c2 receive all the polarized wave components of reflected waves, the accuracy of a second displacement signal generated based on a second IF signal calculated from all the polarized wave components of a reflected wave received by the second receiver 2c2 may be degraded due to the impact of a reflected wave reflected on the reference surface of the reflecting member 3.

In contrast, in this exemplary embodiment, as described above, an aspect is adopted in which the transmitter 2b radiates an electromagnetic wave (transmission signal) including a first direction polarized wave component and a second direction polarized wave component that is different from the first direction polarized wave component and the reflecting member 3 reflects the first direction polarized wave component included in the electromagnetic wave radiated from the transmitter 2b and transmits or absorbs the second direction polarized wave component included in the electromagnetic wave radiated from the transmitter 2b. The first receiver 2c1 receives at least a first direction polarized wave component of a reflected wave reflected on the human body surface of the subject 4 and the reference surface of the reflecting member 3, and the second receiver 2c2 receives a second direction polarized wave component of the reflected wave reflected on the human body surface of the subject 4. With the configuration described above, the second direction polarized wave component of the reflected wave reflected by the reflecting member 3 is suppressed, and the accuracy of the second displacement signal generated based on the second IF signal calculated from the reflected wave of the second direction polarized wave received by the second receiver 2c2 can be increased. In other words, a second displacement signal in which the impact of a reflected wave component from the reflecting member 3 is suppressed can be obtained. Thus, a high-reliability biological signal of the human body of the subject 4 can be obtained.

### Exemplary Embodiment 2

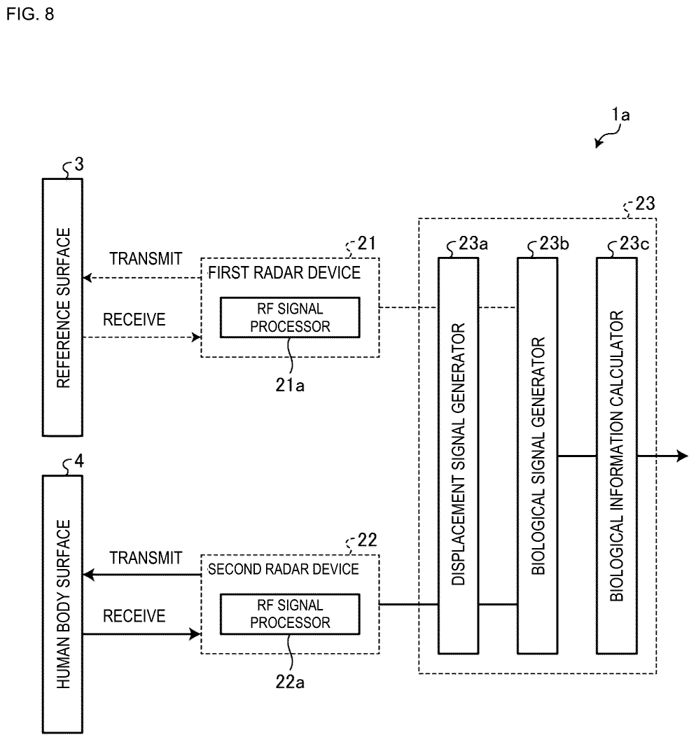

FIG. 8 is a block diagram illustrating a schematic configuration of a biological information detecting apparatus according to Exemplary Embodiment 2. A biological information detecting apparatus 1a according to Exemplary Embodiment 2 includes a first radar device 21, a second radar device 22, a calculator 23, and a reflecting member 3. As with the biological information detecting apparatus 1 according to Exemplary Embodiment 1, the biological information detecting apparatus 1a is applied to, for example, the DMS and is installed inside the seat 5 of a vehicle in which a driver as a subject 4 sits.

The first radar device 21 is, for example, a radar device for the reflecting member. The first radar device 21 radiates, as a vertically polarized wave, an electromagnetic wave toward the reflecting member 3, and receives a reflected wave of the vertically polarized wave reflected on the reference surface of the reflecting member 3. The first radar device 21 includes an RF signal processor 21a inside thereof. The RF signal processor 21a receives the reflected wave received by the first radar device 21 to calculate a first IF signal, converts, with an AD converter, the first IF signal into a digital signal, and outputs the digital signal to the calculator 23.

The second radar device 22 is, for example, a radar device for a human body. The second radar device 22, in synchronization with the first radar device 21, radiates, as a horizontally polarized wave, an electromagnetic wave in the same frequency band as that of the first radar device 21 toward the human body of the subject 4, and receives a reflected wave of the horizontally polarized wave reflected on the human body surface of the subject 4. The second radar device 22 includes an RF signal processor 22a inside thereof. The RF signal processor 22a receives the reflected wave received by the second radar device 22 to calculate a second IF signal, converts, with an AD converter, the second IF signal into a digital signal, and outputs the digital signal to the calculator 23.

In the configuration described above, the reflecting member 3 is based on an aspect similar to the aspect according to Exemplary Embodiment 1 described above with reference to FIG. 3. Specifically, the reflecting member 3 reflects a vertically polarized wave component and transmits a horizontally polarized wave component.

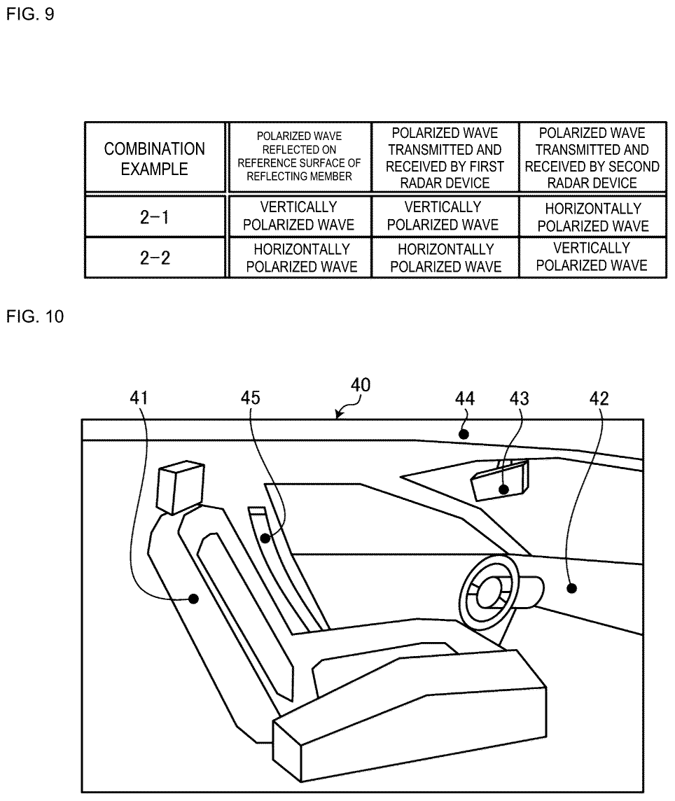

The first radar device 21, the second radar device 22, and the reflecting member 3 are not limited to an aspect described above. FIG. 9 is a diagram illustrating combination examples of a polarized wave of an electromagnetic wave reflected on the reference surface of the reflecting member, a polarized wave of an electromagnetic wave transmitted and received by the first radar device, and a polarized wave of an electromagnetic wave transmitted and received by the second radar device in the biological information detecting apparatus according to Exemplary Embodiment 2.

Specifically, a combination example 2-1 is a combination example in the configuration described above. That is, in an aspect of the combination example 2-1, the first radar device 21 radiates, as a vertically polarized wave, an electromagnetic wave toward the reflecting member 3, and receives a reflected wave of the vertically polarized wave reflected on the reference surface of the reflecting member 3. Furthermore, the second radar device 22 radiates, as a horizontally polarized wave, an electromagnetic wave toward the human body of the subject 4, and receives a reflected wave of the horizontally polarized wave reflected on the human body surface of the subject 4.

In an aspect of a combination example 2-2, the first radar device 21 radiates, as a horizontally polarized wave, an electromagnetic wave toward the reflecting member 3, and receives a reflected wave of the horizontally polarized wave reflected on the reference surface of the reflecting member 3. Furthermore, the second radar device 22 radiates, as a vertically polarized wave, an electromagnetic wave toward the human body of the subject 4, and receives a reflected wave of the vertically polarized wave reflected on the human body surface of the subject 4.

Furthermore, as in Exemplary Embodiment 1, an aspect may be adopted in which the reflecting member 3 including the reference surface is, for example, a slot antenna, a dipole array, a patch antenna array, or the like, and an aspect may be adopted in which the reflecting member 3 is terminated at a predetermined impedance so that components other than a polarized wave received by the first receiver 2c1 can be absorbed.

As described above, an aspect may be adopted in which the first radar device 21 radiates an electromagnetic wave (transmission signal) polarized in a first direction to the reference surface of the reflecting member 3 and the human body surface of the subject 4 and receives a reflected wave of the electromagnetic wave, the second radar device 22 radiates an electromagnetic wave polarized in a second direction that is different from the electromagnetic wave polarized in the first direction to the reference surface of the reflecting member 3 and the human body surface of the subject 4 and receives a reflected wave of the electromagnetic wave, and the reflecting member 3 reflects the electromagnetic wave polarized in the first direction radiated from the first radar device 21 and transmits or absorbs the electromagnetic wave polarized in the second direction radiated from the second radar device 22. An example in which each aspect of the combination example 2-1 illustrated in FIG. 9 is adopted will be described below.

The calculator 23 is a device that has a configuration similar to that of the calculator 2e of the biological information detecting apparatus 1 according to Exemplary Embodiment 1. Specifically, the calculator 23 includes a displacement signal generator 23a, a biological signal generator 23b, and a biological information calculator 23c.

The displacement signal generator 23a generates, based on the first IF signal output from the RF signal processor 21a of the first radar device 21, a first displacement signal. Furthermore, the displacement signal generator 23a generates, based on the second IF signal output from the RF signal processor 22a of the second radar device 22, a second displacement signal.

The biological signal generator 23b isolates the first displacement signal, which is generated based on the first IF signal, from the second displacement signal, which is generated based on the second IF signal, to generate a biological signal of the human body of the subject 4. As in Exemplary Embodiment 1, the present disclosure is not intended to be limited by a technique for isolating the first displacement signal from the second displacement signal.

The first displacement signal is generated based on the first IF signal calculated from the reflected wave reflected on the reference surface of the reflecting member 3 and received by the first receiver 2c1. The first displacement signal is, for example, a component such as vibration caused by road noise or a behavior of the vehicle and is a noise component (see the waveform B in FIG. 6B) with respect to a biological signal to be detected by the biological information detecting apparatus 1 according to the present disclosure.

In contrast, the second displacement signal is generated based on the second IF signal calculated from the reflected wave reflected on the human body surface of the subject 4 and received by the second receiver 2c2. The second displacement signal is a signal component (see the waveform A in FIG. 6A) in which the first displacement signal B illustrated in FIG. 6B is superimposed on a biological signal to be detected by the biological information detecting apparatus 1 according to the present disclosure.

Thus, as in Exemplary Embodiment 1, by isolating the first displacement signal B, which is a noise component, from the second displacement signal A on which the first displacement signal B is superimposed, a biological signal to be detected by the biological information detecting apparatus 1 according to the present disclosure can be extracted (see the waveform C in FIG. 6C).

The biological information calculator 23c calculates, as biological information, vital signs, such as the heart rate, heart rate variability, respiration rate, and depth of respiration, of the human body of the subject 4 from the biological signal generated by the biological signal generator 23b.

A series of steps of a biological information detecting process by the biological information detecting apparatus 1a according to Exemplary Embodiment 2 is similar to that in Exemplary Embodiment 1. The biological information detecting process by the biological information detecting apparatus 1a according to Exemplary Embodiment 2 will be described below with reference to FIG. 7.

First, the biological information detecting apparatus 1a according to Exemplary Embodiment 2 performs, at the same time, the second IF signal acquiring step S101 for acquiring the second IF signal, which is a signal of the human body surface of the subject 4, and the first IF signal acquiring step S102 for acquiring the first IF signal, which is a signal of the reference surface of the reflecting member 3. Specifically, the second IF signal acquiring step S101 and the first IF signal acquiring step S102 are performed, for example, in such a manner that switching between the second IF signal acquiring step S101 and the first IF signal acquiring step S102 is performed every time sampling is performed by the calculator 23. In other words, the second IF signal acquiring step S101 and the first IF signal acquiring step S102 are performed in a time division manner.

The second IF signal acquiring step S101 includes an electromagnetic wave radiating step and a reflected wave receiving step. In the electromagnetic wave radiating step, the second radar device 22 radiates a horizontally polarized electromagnetic wave toward the human body of the subject 4. In the reflected wave receiving step, the second radar device 22 receives, as a human body surface signal, a reflected wave of the horizontally polarized wave reflected on the human body surface of the subject 4.

Furthermore, as with the second IF signal acquiring step S101, the first IF signal acquiring step S102 includes an electromagnetic wave radiating step and a reflected wave receiving step. In the electromagnetic wave radiating step, the first radar device 21 radiates a vertically polarized electromagnetic wave toward the reflecting member 3. In the reflected wave receiving step, the first radar device 21 receives, as a reference surface signal, a reflected wave of the vertically polarized wave reflected on the reference surface of the reflecting member 3.

Next, the biological information detecting apparatus 1a performs the displacement signal generating step S103. In the displacement signal generating step S103, the displacement signal generator 23a generates, from the second IF signal acquired in the second IF signal acquiring step S101, a second displacement signal indicating displacement of the body surface of the human body of the subject 4. Furthermore, the displacement signal generator 23a generates, from the first IF signal acquired in the first IF signal acquiring step S102, a first displacement signal indicating displacement of the reference surface of the reflecting member 3.

Next, the biological information detecting apparatus 1a performs the biological signal generating step S104. In the biological signal generating step S104, the biological signal generator 23b isolates the first displacement signal, which is generated in the displacement signal generating step S103, from the second displacement signal, which is generated in a similar manner in the displacement signal generating step S103. By the biological signal generating step S104, for example, a component such as vibration caused by road noise or a behavior of the vehicle is suppressed, and a biological signal of the human body of the subject 4, which is to be detected by the biological information detecting apparatus 1 according to the present disclosure, is generated. Specifically, for example, the biological signal C illustrated in FIG. 6C includes vital signs such as respiration and heart rate of the subject 4.

Next, the biological information detecting apparatus 1a performs the vital sign acquiring step S105. In the vital sign acquiring step S105, the biological information calculator 23c acquires the vital signs of the human body of the subject 4 from the biological signal generated by the biological signal generator 23b in the biological signal generating step S104. Detailed description of a technique for acquiring vital signs is omitted here, as in Exemplary Embodiment 1. The present disclosure is not intended to be limited by a technique for acquiring vital signs.

With the biological information detecting apparatus 1a and the biological information detecting method according to Exemplary Embodiment 2 described above, by the first radar device 21 and the second radar device 22 that radiate and receive electromagnetic waves in the same frequency bands, electromagnetic waves are radiated and received (output and input) to and from the human body of the subject 4 and the reflecting member 3, and a second displacement signal indicating displacement of the body surface of the human body of the subject 4 and a first displacement signal indicating displacement of the reference surface of the reflecting member 3 are acquired. Then, by the biological signal generator 2g, the first displacement signal is isolated from the acquired second displacement signal, and a biological signal of the human body, in which a noise component superimposed on the second displacement signal is suppressed, is generated.

Thus, unlike a conventional biological information detecting apparatus in which a first sensor and a second sensor that radiate and receive electromagnetic waves at different frequencies are used as radio wave sensors, a biological signal of a human body in which a noise component is suppressed can be obtained by using the first radar device 21 and the second radar device 22 that transmit and receive electromagnetic waves in the same frequency band as radio wave sensors. Hence, with the biological information detecting apparatus 1a according to Exemplary Embodiment 2, an increase of the apparatus scale of the biological information detecting apparatus 1a can be suppressed, and reductions in the size and cost of the biological information detecting apparatus 1a can thus be achieved.

If an aspect is adopted in which the first radar device 21 radiates an electromagnetic wave (transmission signal) of all the polarized waves to the reference surface of the reflecting member 3, the second radar device 22 radiates an electromagnetic wave (transmission signal) of all the polarized waves to the human body surface of the subject 4, the reflecting member 3 reflects the electromagnetic wave of all the polarized waves radiated from the first radar device 21, and the first radar device 21 and the second radar device 22 receive a reflected wave of all the polarized waves, the accuracy of a second displacement signal generated based on a second IF signal calculated from the reflected wave of all the polarized waves received by the second radar device 22 may be degraded due to the impact of a reflected wave reflected on the reference surface of the reflecting member 3.

In contrast, in this exemplary embodiment, as described above, an aspect is adopted in which the first radar device 21 radiates an electromagnetic wave (transmission signal) polarized in the first direction to the reference surface of the reflecting member 3 and the human body surface of the subject 4 and receives a reflected wave of the electromagnetic wave, the second radar device 22 radiates an electromagnetic wave polarized in the second direction that is different from the electromagnetic wave polarized in the first direction to the reference surface of the reflecting member 3 and the human body surface of the subject 4 and receives a reflected wave of the electromagnetic wave, and the reflecting member 3 reflects the electromagnetic wave polarized in the first direction radiated from the first radar device 21 and transmits or absorbs the electromagnetic wave polarized in the second direction radiated from the second radar device 22. With the configuration described above, the reflected wave of the second direction polarized wave reflected by the reflecting member 3 is suppressed, and the accuracy of the second displacement signal generated based on the second IF signal calculated from the reflected wave of the second direction polarized wave transmitted and received by the second radar device 22 can be increased. In other words, a second displacement signal in which the impact of a reflected wave component from the reflecting member 3 is suppressed can be obtained. Thus, as in Exemplary Embodiment 1, a high-reliability biological signal of the human body of the subject 4 can be obtained.

Furthermore, in this exemplary embodiment, each of the first radar device 21 and the second radar device 22 can be arranged at a desired position and is capable of radiating an electromagnetic wave to the human body of the subject 4 and the reflecting member 3 from various angles. Furthermore, since a reflected wave from the human body surface of the subject 4 and the reference surface of the reflecting member 3 can be received at various angles, flexibility in the arrangement of the reflecting member 3 increases. Therefore, arrangement configuration of the biological information detecting apparatus 1a can be designed in a flexible manner.

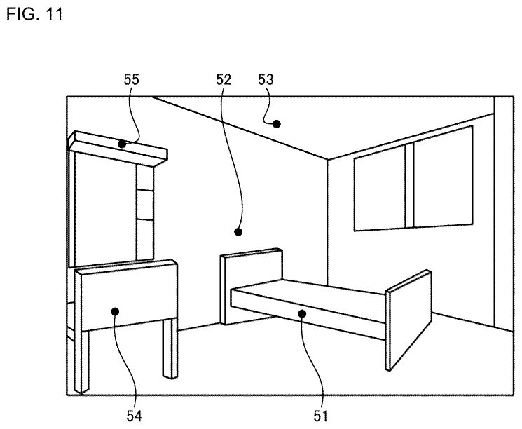

FIG. 10 is a perspective view illustrating an arrangement example of the case where a biological information detecting apparatus according to the present disclosure is applied to a vehicle. In Exemplary Embodiment 1 described above, the case where the radar device 2 and the reflecting member 3 are arranged inside a seat 41 of a vehicle 40 has been described. However, the reflecting member 3 and the radar device 2 may be arranged such that the reflecting member 3 is arranged at a member that picks up motion of the human body between the human body of the subject 4 and the radar device 2 and the radar device 2 is arranged at a position from which electromagnetic waves can be radiated to both the human body and the reflecting member 3 and at which reflected waves from both the human body and the reflecting member 3 can be received. Furthermore, for example, the radar device 2 may be arranged at a dashboard 42, a rear-view mirror 43, a ceiling 44 inside the vehicle, or the like, and the reflecting member 3 may be arranged at a seatbelt 45.

Furthermore, in Exemplary Embodiment 2 described above, the case where the first radar device 21, the second radar device 22, and the reflecting member 3 are arranged inside the seat 41 of the vehicle 40 has been described. However, the reflecting member 3, the first radar device 21, and the second radar device 22 may be arranged such that the reflecting member 3 is arranged at a member that picks up motion of the human body between the human body of the subject 4 and the second radar device 22, the first radar device 21 is arranged at a position from which an electromagnetic wave can be radiated to the human body and at which a reflected wave from the human body can be received, and the second radar device 22 is arranged at a position from which an electromagnetic wave can be radiated to the reflecting member 3 and at which a reflected wave from the reflecting member 3 can be received. Furthermore, for example, the first radar device 21 may be arranged at the dashboard 42, the second radar device 22 may be arranged at the rear-view mirror 43, and the reflecting member 3 may be arranged at the seatbelt 45. Furthermore, the reflecting member 3 may be substituted by a seat heater made of a metal member such as nichrome wire. In this case, for example, an aspect may be adopted in which the first radar device 21, the second radar device 22, and the reflecting member 3 are arranged under the seat surface of the seat 41.

With each of the configurations described above, the vehicle 40 that includes the biological information detecting apparatus 1 or 1a that is capable of obtaining a high-reliability biological signal of a human body while suppressing an increase of the apparatus scale of the biological information detecting apparatus 1 or 1a and reducing the size and cost of the biological information detecting apparatus 1 or 1a can be provided.

Furthermore, the biological information detecting apparatus 1 or 1a according to an exemplary embodiment is not only applicable to a vehicle but is also applicable as, for example, a driver monitoring system for a driver's seat of an aircraft or a train. Furthermore, for example, the biological information detecting apparatus 1 or 1a may be applied to a bed in a medical facility such as a hospital. FIG. 11 is a perspective view illustrating an application example of the case where a biological information detecting apparatus according to the present disclosure is applied to a bed in a medical facility.

In the case where the biological information detecting apparatus 1 according to Exemplary Embodiment 1 described above is applied to a bed in a medical facility, the radar device 2 and the reflecting member 3 may be arranged inside the mattress of a bed 51 in the medical facility. Also in this case, the reflecting member 3 may be arranged at a member that picks up motion of a human body of a subject between the human body and the radar device 2, and the radar device 2 may be arranged at a position from which electromagnetic waves can be radiated to both the human body and the reflecting member 3 and at which reflected waves from both the human body and the reflecting member 3 can be received. Furthermore, for example, the radar device 2 may be arranged at a wall 52, a ceiling 53, a chair 54, a light 55, or the like inside a room of a hospital, and the reflecting member 3 may be arranged at a blanket, which is not illustrated in the drawing, that is placed over the subject.

Furthermore, in the case where the biological information detecting apparatus 1a according to Exemplary Embodiment 2 described above is applied to a bed in a medical facility, the first radar device 21, the second radar device 22, and the reflecting member 3 may be arranged inside the mattress of the bed 51. Also in this case, the reflecting member 3 may be arranged at a member that picks up motion of a human body of a subject between the human body and the second radar device 22, the first radar device 21 may be arranged at a position from which an electromagnetic wave can be radiated to the human body and at which a reflected wave from the human body can be received, and the second radar device 22 may be arranged at a position from which an electromagnetic wave can be radiated to the reflecting member 3 and at which a reflected wave from the reflecting member 3 can be received. For example, the first radar device 21 may be arranged at the wall 52 or the ceiling 53, the second radar device 22 may be arranged at the chair 54 or the light 55, and the reflecting member 3 may be arranged at a blanket, which is not illustrated in the drawing, that is placed over the subject.

With each of the configurations described above, the bed 51 that includes the biological information detecting apparatus 1 or 1a that is capable of obtaining a high-reliability biological signal of a human body while suppressing an increase of the apparatus scale of the biological information detecting apparatus 1 or 1a and reducing the size and cost of the biological information detecting apparatus 1 or 1a can be provided. Furthermore, by arranging the biological information detecting apparatus 1 or 1a inside a room of a hospital as described above, vital signs of, for example, a patient can be detected, and the biological information detecting apparatus 1 or 1a can be used for nursing of, for example, a patient.

The exemplary embodiments described above are intended to facilitate understanding of the present disclosure and are not to be interpreted as limiting the present disclosure. The present disclosure can be modified or improved without departing from the gist of the disclosure, and the present disclosure encompasses equivalents thereof.

Furthermore, the present disclosure may include the following configurations as described above or instead of the above.
(1) A biological information detecting apparatus comprising: a reflecting member that reflects electromagnetic waves; and a radar device that detects a biological signal of a human body, wherein the reflecting member is arranged between the human body and the radar device, wherein the radar device includes a transmitter that radiates an electromagnetic wave toward each of the human body and the reflecting member, and a first receiver that receives a first reflected wave reflected on the human body and the reflecting member, and a second receiver that receives a second reflected wave reflected on the human body.
(2) The biological information detecting apparatus according to (1), wherein the electromagnetic wave radiated by the transmitter includes a first direction polarized wave component and a second direction polarized wave component that is different from the first direction polarized wave component.
(3) The biological information detecting apparatus according to (1) or (2), wherein the reflecting member reflects the first direction polarized wave component and transmits or absorbs the second direction polarized wave component.
(4) The biological information detecting apparatus according to any one of (1) to (3), wherein the first receiver receives at least a reflected first direction polarized wave component of a first reflected wave reflected on the human body and the reflecting member, and the second receiver receives a reflected second direction polarized wave component of a second reflected wave reflected on the human body.
(5) The biological information detecting apparatus according to any one of (1) to (4), comprising: a displacement signal generator that generates, based on the first reflected wave received by the first receiver, a first displacement signal and generates, based on the second reflected wave received by the second receiver, a second displacement signal; and a biological signal generator that isolates the first displacement signal from the second displacement signal to generate a biological signal of the human body.
(6) The biological information detecting apparatus according to any one of (2) to (5), wherein the first direction polarized wave component is polarized in a vertical direction, and the second direction polarized wave component is polarized in a horizontal direction.
(7) The biological information detecting apparatus according to any one of (1) to (6), wherein the reflecting member includes a plurality of reflectors extending in a first direction.
(8) A biological information detecting apparatus comprising: a reflecting member that reflects electromagnetic waves; a first radar device that radiates a first electromagnetic wave polarized in a first direction to the reflecting member and a human body and receives a first reflected wave of the first electromagnetic wave; and a second radar device that radiates a second electromagnetic wave polarized in a second direction that is different from the first electromagnetic wave to the reflecting member and the human body and receives a second reflected wave of the second electromagnetic wave.
(9) The biological information detecting apparatus according to (8), wherein the reflecting member is arranged between the human body and the first radar device, and the reflecting member reflects the first electromagnetic wave and transmits or absorbs the second electromagnetic.
(10) The biological information detecting apparatus according to (8) or (9), comprising: a displacement signal generator that generates, based on the first reflected wave received by the first radar device, a first displacement signal and generates, based on the second reflected wave received by the second radar device, a second displacement signal; and a biological signal generator that isolates the first displacement signal from the second displacement signal to generate a biological signal of the human body.
(11) A vehicle comprising: the biological information detecting apparatus according to (5).
(12) The vehicle according to (11), further comprising circuitry that calculates vital signs of the human body based on the biological signal.
(13) The vehicle according to (12), wherein the vital signs include at least one of heart rate, heart rate variability, respiration rate, and depth of respiration.
(14) A bed comprising: the biological information detecting apparatus according to (5).
(15) The vehicle according to (14), further comprising circuitry that calculates vital signs of the human body based on the biological signal.
(16) The vehicle according to (15), wherein the vital signs include at least one of heart rate, heart rate variability, respiration rate, and depth of respiration.
(17) A biological information detecting method comprising: radiating a first electromagnetic wave including a first direction polarized wave component and a second direction polarized wave component that is different from the first direction polarized wave component toward a human body; receiving a first reflected wave of the second direction polarized wave component, out of the first direction polarized wave component and the second direction polarized wave component included in the first electromagnetic wave radiated; radiating a second electromagnetic wave including a first direction polarized wave component and a second direction polarized wave component toward a reflecting member that reflects electromagnetic waves; receiving a second reflected wave of the first direction polarized wave component, out of the first direction polarized wave component and the second direction polarized wave component included in the second electromagnetic wave radiated; and generating a biological signal of the human body based on the first reflected wave received and the second reflected wave received.
(18) The biological information detecting method according to (17), wherein to generate the biological signal of the human body the method further comprises: generating, from the second reflected wave received, a second displacement signal and generating, from the first reflected wave received, a first displacement signal; and isolating the first displacement signal from the second displacement signal to generate the biological signal of the human body.
(19) A biological information detecting method comprising: radiating a first electromagnetic wave polarized in a first direction toward a reflecting member that reflects the first electromagnetic wave and radiating a second electromagnetic wave polarized in a second direction that is different from the first electromagnetic wave polarized in the first direction toward a human body; receiving a first reflected wave of the first electromagnetic wave and receiving a second reflected wave of the second electromagnetic wave; and generating a biological signal of the human body based on the first reflected wave received and the second reflected wave received.
(20) The biological information detecting method according to (19), wherein to generate the biological signal of the human body the method further comprises: generating, from the second reflected wave of the second electromagnetic wave p, a second displacement signal and generating, from the first reflected wave of the first electromagnetic wave, a first displacement signal; and isolating the first displacement signal from the second displacement signal to generate the biological signal of the human body.

### REFERENCE SIGNS LIST

1, 1a biological information detecting apparatus
2 radar device
2a RF signal generator
2b transmitter
2c1 first receiver
2c2 second receiver
2d RF signal processor
2e calculator
2f displacement signal generator
2g biological signal generator
2h biological information calculator
3 reflecting member
3a reflector
4 subject
5 seat
5a inner material
5b surface material
21 first radar device
21a RF signal processor
22 second radar device
22a RF signal processor
23 calculator
23a displacement signal generator
23b biological signal generator
23c biological information calculator
40 vehicle
51 bed

1. A biological information detecting apparatus comprising:
a reflecting member that reflects electromagnetic waves; and
a radar device that detects a biological signal of a human body,
wherein the reflecting member is arranged between the human body and the radar device,
wherein the radar device includes
a transmitter that radiates an electromagnetic wave toward each of the human body and the reflecting member, and
a first receiver that receives a first reflected wave reflected on the human body and the reflecting member, and
a second receiver that receives a second reflected wave reflected on the human body.

2. The biological information detecting apparatus according to claim 1, wherein the electromagnetic wave radiated by the transmitter includes a first direction polarized wave component and a second direction polarized wave component that is different from the first direction polarized wave component.

3. The biological information detecting apparatus according to claim 2, wherein the reflecting member reflects the first direction polarized wave component and transmits or absorbs the second direction polarized wave component.

4. The biological information detecting apparatus according to claim 3, wherein the first receiver receives at least a reflected first direction polarized wave component of a first reflected wave reflected on the human body and the reflecting member, and
the second receiver receives a reflected second direction polarized wave component of a second reflected wave reflected on the human body.

5. The biological information detecting apparatus according to claim 4, comprising:
a displacement signal generator that generates, based on the first reflected wave received by the first receiver, a first displacement signal and generates, based on the second reflected wave received by the second receiver, a second displacement signal; and
a biological signal generator that isolates the first displacement signal from the second displacement signal to generate a biological signal of the human body.

6. The biological information detecting apparatus according to claim 2, wherein the first direction polarized wave component is polarized in a vertical direction, and the second direction polarized wave component is polarized in a horizontal direction.

7. The biological information detecting apparatus according to claim 1, wherein the reflecting member includes a plurality of reflectors extending in a first direction.

8. A biological information detecting apparatus comprising:
a reflecting member that reflects electromagnetic waves;
a first radar device that radiates a first electromagnetic wave polarized in a first direction to the reflecting member and a human body and receives a first reflected wave of the first electromagnetic wave; and
a second radar device that radiates a second electromagnetic wave polarized in a second direction that is different from the first electromagnetic wave to the reflecting member and the human body and receives a second reflected wave of the second electromagnetic wave.

9. The biological information detecting apparatus according to claim 8, wherein the reflecting member is arranged between the human body and the first radar device, and
the reflecting member reflects the first electromagnetic wave and transmits or absorbs the second electromagnetic.

10. The biological information detecting apparatus according to claim 9, comprising:
a displacement signal generator that generates, based on the first reflected wave received by the first radar device, a first displacement signal and generates, based on the second reflected wave received by the second radar device, a second displacement signal; and
a biological signal generator that isolates the first displacement signal from the second displacement signal to generate a biological signal of the human body.

11. A vehicle comprising:
the biological information detecting apparatus according to claim 5.

12. The vehicle according to claim 11, further comprising circuitry that calculates vital signs of the human body based on the biological signal.

13. The vehicle according to claim 12, wherein the vital signs include at least one of heart rate, heart rate variability, respiration rate, and depth of respiration.

14. A bed comprising:
the biological information detecting apparatus according to claim 5.

15. The vehicle according to claim 14, further comprising circuitry that calculates vital signs of the human body based on the biological signal.

16. The vehicle according to claim 15, wherein the vital signs include at least one of heart rate, heart rate variability, respiration rate, and depth of respiration.

17. A biological information detecting method comprising:
radiating a first electromagnetic wave including a first direction polarized wave component and a second direction polarized wave component that is different from the first direction polarized wave component toward a human body;
receiving a first reflected wave of the second direction polarized wave component, out of the first direction polarized wave component and the second direction polarized wave component included in the first electromagnetic wave radiated;
radiating a second electromagnetic wave including a first direction polarized wave component and a second direction polarized wave component toward a reflecting member that reflects electromagnetic waves;
receiving a second reflected wave of the first direction polarized wave component, out of the first direction polarized wave component and the second direction polarized wave component included in the second electromagnetic wave radiated; and
generating a biological signal of the human body based on the first reflected wave received and the second reflected wave received.

18. The biological information detecting method according to claim 17, wherein to generate the biological signal of the human body the method further comprises:
generating, from the second reflected wave received, a second displacement signal and generating, from the first reflected wave received, a first displacement signal; and
isolating the first displacement signal from the second displacement signal to generate the biological signal of the human body.

19. A biological information detecting method comprising:
radiating a first electromagnetic wave polarized in a first direction toward a reflecting member that reflects the first electromagnetic wave and radiating a second electromagnetic wave polarized in a second direction that is different from the first electromagnetic wave polarized in the first direction toward a human body;
receiving a first reflected wave of the first electromagnetic wave and receiving a second reflected wave of the second electromagnetic wave; and
generating a biological signal of the human body based on the first reflected wave received and the second reflected wave received.

20. The biological information detecting method according to claim 19, wherein to generate the biological signal of the human body the method further comprises:
generating, from the second reflected wave of the second electromagnetic wave p, a second displacement signal and generating, from the first reflected wave of the first electromagnetic wave, a first displacement signal; and
isolating the first displacement signal from the second displacement signal to generate the biological signal of the human body.
